(19) United States
(12) Patent Application Publication
(10) Pub. No.: US 2004/0092843 A1
(43) Pub. Date: May 13, 2004

(54) Device and method for measuring fetal blood pH

(76) Inventors: Doron Kreiser, Herzlia (IL); Amir Belson, Cupertino, CA (US)

Correspondence Address: LEARY & ASSOCIATES, 3900 NEWPARK MALL RD., THIRD FLOOR, SUITE 317, NEWARK, CA, 94560, US

(21) Appl. No.: 10/082,808

(22) Filed: Feb. 22, 2002

Related U.S. Application Data
(60) Provisional application No. 60/270,669, filed on Feb. 22, 2001.

(51) Int. Cl. A61B 5/00; B65D 81/00

(52) U.S. Cl. 600/576; 600/583

(57) ABSTRACT

A hand-mounted device for obtaining a sample of blood from the scalp of a fetus and for measuring the pH of the blood sample includes a lancet for incising the scalp of the fetus, a capillary for obtaining a drop-sized sample of blood and a pH electrode within the capillary for analyzing the pH of the blood. Optionally, a motorized actuator may be provided for automatically advancing and withdrawing the lancet and the capillary. The pH measuring device includes an isolating mechanism, in the form of an everting bell-shaped member, an inflatable balloon member or an expandable funnel member, and a flushing/aspiration lumen for clearing the distal region of the device of amniotic fluid while in use.

### CROSS REFERENCE TO OTHER APPLICATIONS

The present application claims the benefit of U.S. Provisional Patent Application 60/270,669, filed on Feb. 22, 2001.

### FIELD OF THE INVENTION

The present invention relates generally to devices and methods for obtaining a sample of blood from the scalp of a fetus and for measuring the pH of the blood sample. More particularly, it relates to a device for measuring fetal blood pH that is mounted on a physician's hand and incorporated into a glove, finger cot or ring for convenience of use.

### BACKGROUND OF THE INVENTION

Fetal blood pH is an important indicator of fetal stress during labor and delivery. The following U.S. patents describe devices and methods of measuring fetal blood pH: U.S. Pat. No. 6,058,321 Instrument for continuously monitoring fetal heart rate and intermittently monitoring fetal blood pH and method of use; and U.S. Pat. No. 4,441,510 Method and apparatus for fetal pH scalp studies. The following U.S. patent discloses a finger-tip applicator for placement of an ECG electrode on the scalp of a fetus: U.S. Pat. No. 4,244,375 Transcutaneous electrode with finger operative attachment assembly. These patents and all other patents and patent applications referred to herein are hereby incorporated by reference in their entirety.

The hand-mounted device for measuring fetal blood pH is adaptable for use with the obstetrical imaging system and integrated fetal vacuum extraction system described in commonly-owned and copending U.S. patent application Ser. No. 10/024,656, filed on Dec. 17, 2001, which is incorporated by reference in its entirety.

### SUMMARY OF THE INVENTION

The present invention takes the form of a hand-mounted device for obtaining a sample of blood from the scalp of a fetus during parturition and for measuring the pH of the blood sample. The pH measuring device can be mounted on a surgical glove, a finger cot or on a ring. The pH measuring device includes a lancet that is slidably received within a lancet guide tube and a capillary that is slidably received within a capillary guide tube. The lancet guide tube and the capillary guide tube extend from a location near the distal end of one of the fingers of the glove, for example the index finger, to the proximal end of the glove located at the user's wrist. A lancet actuator rod is connected to the lancet and extends proximally through the lancet guide tube to an actuator at the proximal end of the lancet guide tube. A capillary actuator rod or tube is connected to the capillary and extends proximally through the capillary guide tube to a capillary actuator at the proximal end of the capillary guide tube. Preferably, the guide tubes and the actuator rods are sufficiently flexible that they will not interfere with the flexibility of the glove or the dexterity of the operator's hand. Optionally, a motorized actuator may be provided for sequentially advancing and withdrawing the lancet and the capillary automatically in order to facilitate operation of the device.

In one embodiment, the capillary is configured with a small bulb at its distal end with a pH electrode mounted within the bulb. The electrode leads extend through the capillary guide tube alongside the capillary actuator rod and connect to an electronic pH meter. Alternatively, the capillary may be a simple tube that is removable through the proximal end of the capillary guide tube so that the blood sample can be placed into a pH meter.

The pH measuring device preferably includes a mechanism for isolating the distal region of the device from amniotic fluid while in use. The mechanism for isolating the distal region of the device may be in the form of an everting bell-shaped member, an inflatable balloon member or an expandable funnel member.

In operation, the pH measuring device is mounted on the physician's hand similar to a normal surgical glove. When the physician wishes to determine the blood pH of the fetus, the finger of the glove with the pH measuring device is placed in proximity to the scalp or other presenting part of the fetus. The isolation device is actuated to isolate a region between the scalp and the distal end of the pH measuring device. If necessary, the region can be flushed with saline solution or with air to clear away amniotic fluid and/or the region can be cleaned with one of the other fingers on the operator's hand. The lancet is advanced to incise the scalp, then the lancet is withdrawn into the lancet guide tube. The capillary is then advanced toward the point of the incision to obtain a drop-sized sample of blood and the capillary is withdrawn into the capillary guide tube. Optionally, the lancet and the capillary may be advanced and withdrawn automatically by a motorized actuator. A pH electrode in the device analyzes the pH of the blood sample and the value of the pH is displayed on an electronic pH meter. Alternatively, the capillary can be withdrawn and placed in a separate pH meter for analysis.

### BRIEF DESCRIPTION OF THE DRAWINGS

FIG. 1 shows a hand-mounted device for obtaining a sample of blood from the scalp of a fetus and for measuring the pH of the blood sample.

FIG. 2 is an enlarged detail view of the hand-mounted pH measuring device of FIG. 1.

FIG. 3 is an enlarged detail view of a capillary bulb with a pH electrode.

FIG. 4 shows an everting bell-shaped member for isolating the distal region of the pH measuring device.

FIG. 5 shows an enlarged cross section of the everting bell-shaped member of FIG. 4.

FIG. 6 shows an inflatable balloon member for isolating the distal region of the pH measuring device.

FIG. 7 shows an expandable funnel member for isolating the distal region of the pH measuring device.

### DETAILED DESCRIPTION OF THE INVENTION

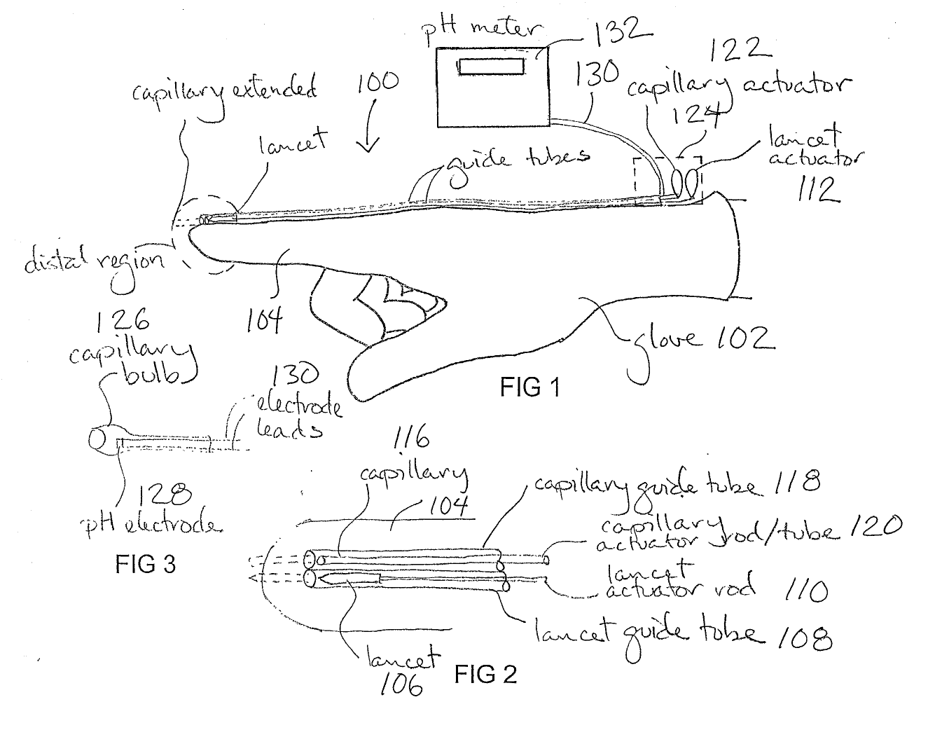

FIG. 1 shows a hand-mounted device 100 for obtaining a sample of blood from the scalp of a fetus and for measuring the pH of the blood sample. The pH measuring device 100 can be mounted on a surgical glove 102, as shown, or, alternatively, it can be mounted on a finger cot or on a ring. The pH measuring device 100 includes a lancet 106 that is slidably received within a lancet guide tube 108 and a capillary 116 that is slidably received within a capillary guide tube 118. The lancet guide tube 108 and the capillary guide tube 118 extend from a location near the distal end of one of the fingers 104 of the glove 102, for example the index finger, to the proximal end of the glove located at the user's wrist. A lancet actuator rod 110 is connected to the lancet 106 and extends proximally through the lancet guide tube 108 to a lancet actuator 112 at the proximal end of the lancet guide tube 108. A capillary actuator rod 120 or tube is connected to the capillary 116 and extends proximally through the capillary guide tube 118 to a capillary actuator 122 at the proximal end of the capillary guide tube 118. Preferably, the guide tubes and the actuator rods are sufficiently flexible that they will not interfere with the flexibility of the glove or the dexterity of the operator's hand.

FIG. 2 is an enlarged detail view of the distal end of the hand-mounted pH measuring device 100 of FIG. 1 shown from the top of the index finger 104 of the glove. The lancet 106 has a sharp point capable of puncturing or incising the scalp of the fetus. The lancet 106 is extendable beyond the distal end of the lancet guide tube 108 and past the end of the finger 104 of the glove. The capillary 116 is a small diameter tube with an internal diameter sized to draw a blood sample into it by capillary action. The capillary 116 is extendable beyond the distal end of the capillary guide tube 118 and past the end of the finger 104 of the glove.

In one particularly preferred embodiment shown in an enlarged detail view in FIG. 3, the capillary 116 is configured with a small bulb 126 at its distal end with a pH electrode 128 mounted within the bulb. The electrode leads 130 extend through the capillary guide tube 118 alongside the capillary actuator rod 120 and connect to an electronic pH meter 132.

In an alternate embodiment, the capillary 116 may be a simple tube constructed without an integral pH electrode. The capillary 116 is removable through the proximal end of the capillary guide tube 118 so that the blood sample can be placed into a pH meter.

Preferably, the pH measuring device 100 includes a mechanism for isolating the distal region of the device from amniotic fluid while in use. Various mechanisms for isolating the distal region of the device are described below.

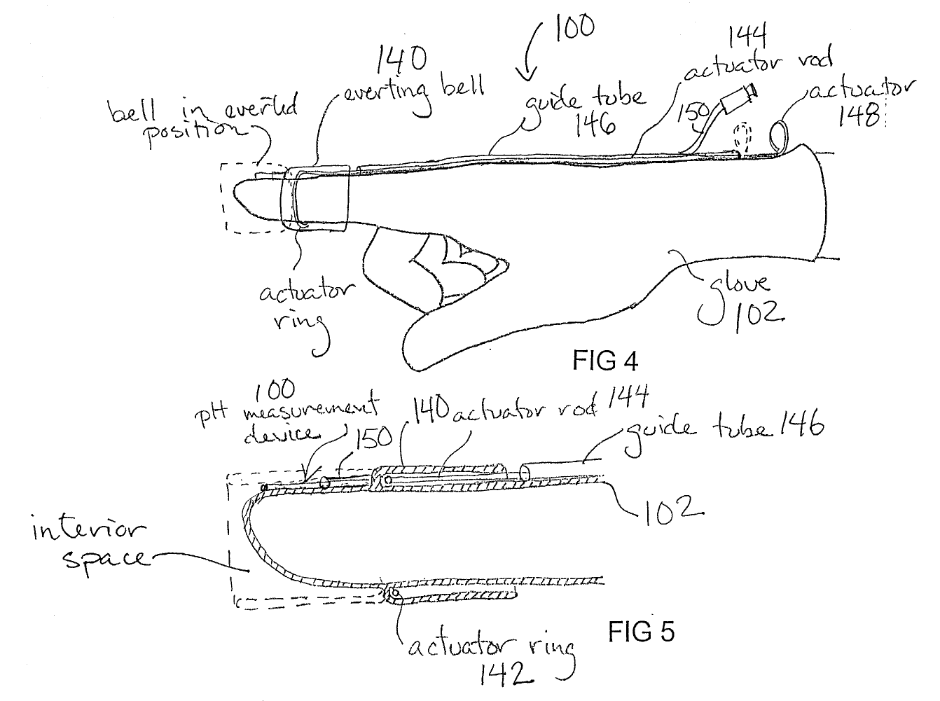

FIG. 4 shows an everting bell-shaped member 140 for isolating the distal region of the pH measuring device 100. FIG. 5 shows an enlarged cross section of the everting bell-shaped member 140 of FIG. 4. The bell-shaped member 140 can be molded integrally with the surgical glove 102, as shown in FIG. 5, or it can be constructed as a separate piece. The bell-shaped member 140 is movable from a retracted position to an extended position by everting the bell-shaped member 140. An actuator ring 142 for everting the bell-shaped member 140 is connected to an actuator rod 144 that extends through a guide tube 146 to an actuator 148 on the proximal end of the device. Optionally, the pH measuring device 100 may also include a tube 150 with a lumen that is open to the interior space of the bell-shaped member 140 when it is in the extended position for flushing the interior space of the bell-shaped member 140 with saline solution or air and for applying suction to aspirate the interior space.

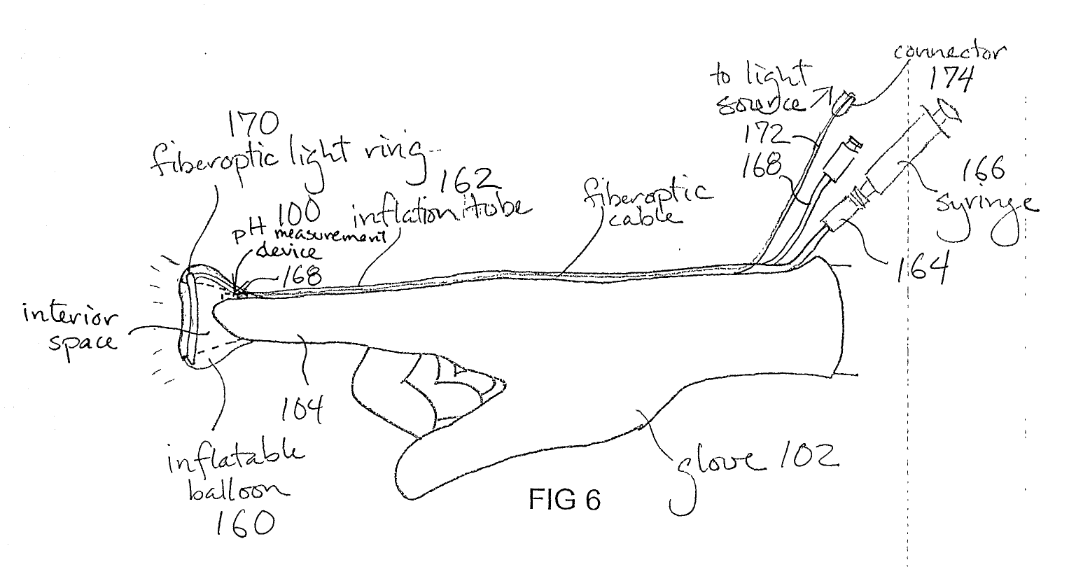

FIG. 6 shows an inflatable balloon member 160 for isolating the distal region of the pH measuring device 100. The inflatable balloon member is attached to the finger 104 of the glove 102 and surrounds the distal end of the pH measuring device 100. The balloon member 160 is connected to an inflation lumen within an inflation tube 162 that extends proximally to a connector 164 for attachment to a syringe 166 or the like. When inflated, the inflatable balloon member 160 expands to a funnel-shape that surrounds the distal end of the pH measuring device 100 and the operator's fingertip. The pH measuring device 100 may optionally include a flushing/suction tube 168 with a lumen that is open to the interior space of the funnel-shaped balloon member 160 when it is in the inflated state. Optionally, the balloon member 160 may also include a fiberoptic light emitting ring 170 that is connected to a fiberoptic cable 172 with a connector 174 adapted for connection to a light source.

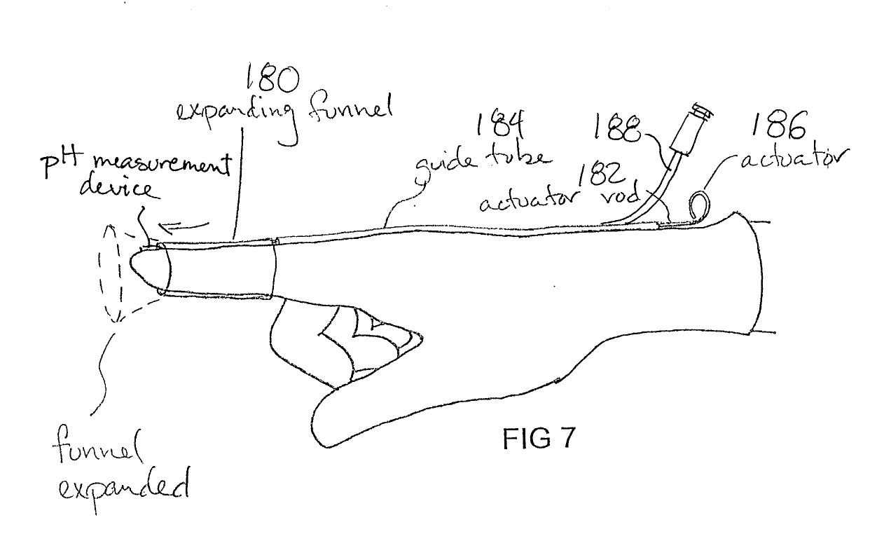

FIG. 7 shows an expandable funnel member 180 for isolating the distal region of the pH measuring device 100. The funnel member 180 surrounds the operator's finger and is slidable from a retracted position to an extended position by an actuator rod 182 that extends through a guide tube 184 to an actuator 186 on the proximal end of the device. Preferably, the tubular funnel member 180 is constructed so that it expands to form a funnel shape as it is advanced distally to isolate the region around the distal end of the pH measuring device 100. Again, the pH measuring device 100 may optionally include a flushing/suction tube 188 with a lumen that is open to the interior space of the funnel member 180 when it is in the extended position.

In operation, the pH measuring device 100 is mounted on the physician's hand similar to a normal surgical glove. The pH measuring device 100 is flexible enough that it does not interfere with the physician's ability to palpate the fetus for diagnosis. When the physician wishes to determine the blood pH of the fetus, the finger of the glove with the pH measuring device 100 is placed in proximity to the scalp of the fetus, preferably at a location away from the fontanels or the sutures of the skull. The isolation device is actuated to isolate a region between the scalp and the distal end of the pH measuring device 100. If necessary, the region can be flushed with saline solution or with air through the optional flushing/suction lumen to clear away amniotic fluid and/or the region can be cleaned with one of the other fingers on the operator's hand. The lancet 106 is advanced to incise the scalp, then the lancet 106 is withdrawn into the lancet guide tube 108. The capillary 116 is then advanced toward the point of the incision to obtain a drop-sized sample of blood and the capillary 116 is withdrawn into the capillary guide tube 118. In the preferred embodiment, the pH electrode 128 analyzes the pH of the blood sample and the value of the pH is displayed on the electronic pH meter 132.

Alternatively, the capillary 116 can be withdrawn and placed in a separate pH meter for analysis. Preferably, this procedure is performed under direct visualization using the obstetrical imaging system as described in U.S. patent application Ser. No. 10/024,656, filed on Dec. 17, 2001.

In an alternate embodiment of the pH measuring device 100, the lancet 106 and the capillary 116 can be actuated by a motorized actuator 124, shown in dashed lines in FIG. 1, so that they advance and withdraw automatically in order to facilitate operation of the device.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof. For example, many of the features described can be used together in combinations other than those explicitly described.

What is claimed is:

1. A device for measuring fetal blood pH, comprising:
means for mounting the device to an operator's hand;
a lancet extendable from a distal end of the device for incising tissue; and
a capillary extendable from the distal end of the device for collecting a sample of blood for pH analysis.

2. The device for measuring fetal blood pH of claim 1, wherein the means for mounting the device to an operator's hand is in the form of a surgical glove to which the device is mounted.

3. The device for measuring fetal blood pH of claim 1, wherein the means for mounting the device to an operator's hand is in the form of a finger cot to which the device is mounted.

4. The device for measuring fetal blood pH of claim 1, wherein the means for mounting the device to an operator's hand is in the form of a finger ring to which the device is mounted.

5. The device for measuring fetal blood pH of claim 1, wherein the lancet is slidably mounted in a lancet guide tube.

6. The device for measuring fetal blood pH of claim 5, wherein the lancet is connected by a lancet actuator rod to a lancet actuator located at a proximal end of the lancet guide tube.

7. The device for measuring fetal blood pH of claim 1, wherein the capillary is slidably mounted in a capillary guide tube.

8. The device for measuring fetal blood pH of claim 7, wherein the capillary is connected by a capillary actuator rod to a capillary actuator located at a proximal end of the capillary guide tube.

9. The device for measuring fetal blood pH of claim 7, wherein the capillary is configured to be withdrawn from a proximal end of the capillary guide tube.

10. The device for measuring fetal blood pH of claim 1, wherein the capillary has a distal end configured as a capillary bulb.

11. The device for measuring fetal blood pH of claim 10, further comprising a pH electrode positioned within the capillary bulb.

12. The device for measuring fetal blood pH of claim 1, further comprising means for isolating a distal region of the pH measuring device from a surrounding region.

13. The device for measuring fetal blood pH of claim 12, wherein the means for isolating a distal region of the pH measuring device from a surrounding region is in the form of an everting bell-shaped member.

14. The device for measuring fetal blood pH of claim 12, wherein the means for isolating a distal region of the pH measuring device from a surrounding region is in the form of an inflatable balloon member.

15. The device for measuring fetal blood pH of claim 12, wherein the means for isolating a distal region of the pH measuring device from a surrounding region is in the form of an expandable funnel member.

16. The device for measuring fetal blood pH of claim 1, further comprising a motorized actuator for sequentially advancing the lancet and the capillary.

17. The device for measuring fetal blood pH of claim 1, further comprising a light source for illuminating a region distal to the device.

18. A device for measuring fetal blood pH, comprising:
a lancet guide tube;
a lancet slidably mounted in the lancet guide tube and connected by a lancet actuator rod to a lancet actuator located at a proximal end of the lancet guide tube, wherein the lancet is selectively extendable from a distal end of the lancet guide tube for incising tissue;
capillary guide tube;
a capillary slidably mounted in the capillary guide tube and connected by a capillary actuator rod to a capillary actuator located at a proximal end of the capillary guide tube, wherein the capillary is selectively extendable from a distal end of the capillary guide tube for collecting a sample of blood for pH analysis; and
means for mounting the lancet guide tube and the capillary guide tube to an operator's hand.

19. The device for measuring fetal blood pH of claim 18, further comprising means for isolating a distal region of the pH measuring device from a surrounding region.

20. The device for measuring fetal blood pH of claim 18, further comprising a motorized actuator for sequentially advancing the lancet and the capillary.

21. A method for measuring fetal blood pH, comprising:
inserting a hand-mounted pH device in proximity of a presenting part of a fetus in parturition;
isolating a region adjacent to a distal end of the pH device from a surrounding region;
advancing a lancet from the distal end of the pH device and incising tissue in the presenting part of the fetus; and
advancing a capillary from the distal end of the pH device and collecting a sample of blood from the incised tissue for pH analysis.
